(19) United States
(12) Patent Application Publication
(10) Pub. No.: US 2024/0242803 A1
(43) Pub. Date: Jul. 18, 2024

(54) MEDICAL LEARNING SYSTEM, MEDICAL LEARNING METHOD, AND STORAGE MEDIUM

(71) Applicant: Canon Medical Systems Corporation, Otawara-shi (JP)

(72) Inventors: Yusuke KANO, Nasushiobara (JP); Satoshi IKEDA, Yaita (JP)

(73) Assignee: Canon Medical Systems Corporation, Otawara-shi (JP)

(21) Appl. No.: 18/410,369

(22) Filed: Jan. 11, 2024

(30) Foreign Application Priority Data
Jan. 18, 2023 (JP) 2023-006029

(51) Int. Cl. G16H 20/00; G16H 10/60; G16H 50/70

(52) U.S. Cl. CPC G16H 20/00; G16H 10/60; G16H 50/70

(57) ABSTRACT

A medical learning system according to an embodiment includes processing circuitry. The processing circuitry acquires a first inference model that infers a treatment action of a target medical care provider based on a state of a patient. The processing circuitry acquires treatment progress data relating to a target patient. The processing circuitry generates a second inference model in conformity with the target patient by updating the first inference model based on the treatment progress data.

### CROSS-REFERENCE TO RELATED APPLICATION

This application is based upon and claims the benefit of priority from Japanese Patent Application No. 2023-006029, filed Jan. 18, 2023, the entire contents of which are incorporated herein by reference.

### FIELD

Embodiments described herein relate generally to a medical learning system, a medical learning method, and a storage medium.

### BACKGROUND

The realization of clinical decision support (CDS) and knowledge acquisition based on accumulated medical data has been attempted. The technology for training an artificial intelligence (AI) model with data on doctors' actions can be considered an example of this. This AI model infers a treatment action that a doctor should take from the state of a patient. Specifically, it is to be expected that an AI model generating policies near-identical to those of a human doctor can be achieved by applying, to a doctor's actual action data, techniques of initializing a policy function through behavior cloning and updating the policy function under a Kullback-Leibler (KL) distance restriction between policies before and after updating. However, since this involves the averaging of policies of a plurality of doctors, specific characteristics pertaining to each doctor's expertise and sense of values tend to be lost. Furthermore, a large knowledge gap between an AI model and each doctor causes interpretability to deteriorate.

### BRIEF DESCRIPTION OF THE DRAWINGS

FIG. 1 is a diagram showing an example of a network configuration of a medical learning system according to an embodiment.

FIG. 2 is a diagram showing a configuration example of a medical learning apparatus according to a first embodiment.

FIG. 3 is a diagram schematically showing a processing sequence for medical learning processing by a medical learning apparatus according Example 1 of the first embodiment.

FIG. 4 is a diagram showing an input and an output of a doctor model.

FIG. 5 is a diagram showing an input and an output of an improved doctor model.

FIG. 6 is a diagram schematically showing a processing sequence for medical learning processing by a medical learning apparatus according to Example 1 of a second embodiment.

FIG. 7 is a diagram showing an input and an output of a patient model.

FIG. 8 is a diagram showing a configuration example for a medical learning apparatus according to the second embodiment.

FIG. 9 is a diagram schematically showing an example for a search process in the second embodiment.

FIG. 10 is a diagram showing a configuration example for a medical learning apparatus according to a third embodiment.

FIG. 11 is a drawing schematically showing a management process for an improved doctor model.

FIG. 12 is a diagram showing an example of a network architecture for a doctor model according to an development example.

FIG. 13 is a diagram showing an example of a network architecture for a patient model according to the development example.

FIG. 14 is a diagram schematically showing a search process in the development example.

### DETAILED DESCRIPTION

A medical learning system of an embodiment includes a model acquisition unit, a data acquisition unit, and a generation unit. A model acquisition unit acquires a first inference model that infers a treatment action of a target medical care provider based on the state of a patient. The data acquisition unit acquires medical diagnosis and treatment data relating to a target patient. The generation unit generates a second inference model by updating the first inference model based on the medical diagnosis and treatment data.

Hereinafter, a medical learning system, a medical learning method, and a storage medium according to the present embodiment will be described with reference to the accompanying drawings.

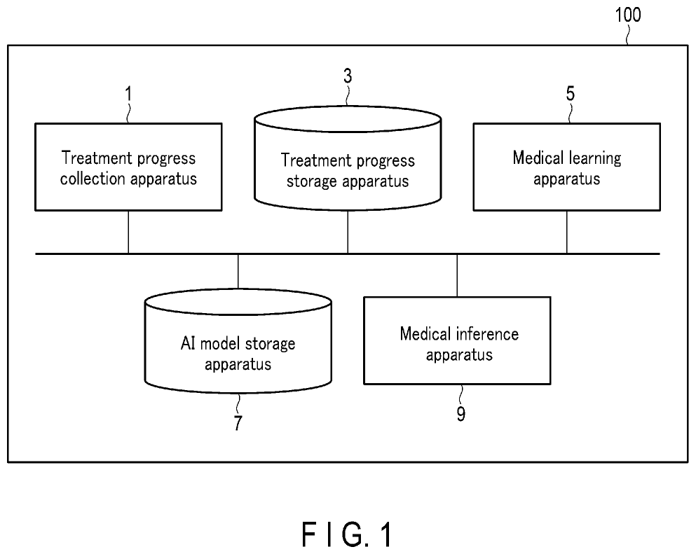

FIG. 1 is a diagram showing an example of a network configuration of a medical learning system 100 according to the present embodiment. As shown in FIG. 1, the medical learning system 100 includes a treatment progress collection apparatus 1, a treatment progress storage device 3, a medical learning apparatus 5, an AI model storage device 7, and a medical inference apparatus 9. The treatment progress collection apparatus 1, the treatment progress storage device 3, the medical learning apparatus 5, and the AI model storage device 7, and the medical inference apparatus 9 are connected by wire or wirelessly to each other in such a manner that they can communicate with each other. The medical learning system 100 may include one or more treatment progress collection apparatuses 1, treatment progress storage devices 3, medical learning apparatuses 5, AI model storage devices 7, and medical inference apparatuses 9, respectively.

The treatment progress collection apparatus 1 collects data representing progress of medical diagnosis and treatment (hereinafter “medical diagnosis and treatment data”) relating to a plurality of medical care recipients and a plurality of medical care providers. A medical care recipient is a person who receives a treatment action, and is herein assumed to be a patient. A medical care provider is a person who conducts a medical diagnosis and implements a treatment action, typically a doctor, a nurse, a radiology technician, a pharmacist, a physical therapist, or a care worker, and is hereinafter assumed to be a doctor. The treatment progress data means sequential data of samples including a state sₜⁱ of a patient i at a time point t, a doctor j's treatment action aₜ^{(i,j)} taken for the patient i in the state of sₜⁱ, a state sₜ₊₁ⁱ of the patient i at a next time point t+1 after the patient receives the treatment action aₜ^{(i,j)}, and a reward rₜⁱ denoting a treatment effect in the patient i with respect to the treatment action aₜ^{(i,j)}. The reward rₜⁱ is not essential, and it suffices merely to include it when needed to generate various models. A time point t may be defined by an absolute time or a time difference from a reference time.

A state is data represented by a blood pressure, a heart rate, a blood glucose level, SpO2, and other biometric information. A blood pressure, a heart rate, a blood glucose level, SpO2, and etc., which are elements of a state, may be referred to as a “state element”. A state or a state element is collected by a biological information collecting device selected depending on a type of biometric information. A state or a state element may not only be data collected by a biometric information collecting device but also a medical image collected by various medical image diagnosis apparatuses, an image measurement value measured by an image processing apparatus based on the medical image, or the like. A state or a state element may be data acquired through a medical examination by interview, etc. conducted by a doctor j for a patient i. A state may be represented by a scalar quantity that includes one of the above state elements or a vector quantity or a matrix quantity that includes a combination of a plurality of state elements. A state is represented by numbers, letters, symbols, etc. Examples of the medical diagnosis and treatment process collection apparatus 1 that collects a state include a biometric information collecting device, a medical image diagnosis apparatus, a medical image processing apparatus, and a computer terminal used by a doctor during medical diagnosis and treatment, etc., according to various elements of a state.

A treatment action is data represented by a specific medical practice, such as a medication treatment, a surgical operation, radiation therapy, etc. A specific medical practice, such as a medication treatment, a surgical operation, radiation therapy, etc., which constitutes an element of a treatment action, may be referred to as a “treatment action element”. A treatment action may be represented by a scalar quantity that includes one of the above treatment action element or a vector quantity or a matrix quantity that includes a combination of a plurality of the treatment action elements. A treatment action is represented by numbers, letters, symbols, etc. Examples of the treatment progress collection apparatus 1 that collects treatment actions include a computer terminal, etc. used by a doctor during a medical diagnosis and treatment.

A reward is data for evaluating a treatment action, for example data represented by a clinical outcome, a patient report outcome, an economic outcome, etc. A clinical outcome, a patient report outcome, an economic outcome, etc., which are elements of a reward, may be referred to as a “reward element”. Examples of a clinical outcome include a morbidity rate (including whether a patient is affected by a disease or not), a five-year survival rate (including whether a patient survived or not), a complication rate (including whether or not a patient suffers from a complication), a re-admission rate (including whether a patient is re-hospitalized or not), an examination value (or a level of improvement in an examination value), a degree of independence in a patient's daily life, etc. Examples of a patient report outcome include a subjective symptom, a subjectively observed health state, a level of satisfaction toward a treatment, and a subjectively observed happiness level. Examples of an economic outcome include medical bills, committed medical resources, the number of hospitalized days, etc. A reward may be represented by a scalar quantity corresponding to one of the above reward elements or a vector quantity or a matrix quantity that includes a combination of a plurality of reward elements. A reward is represented by numbers, letters, symbols, etc. Examples of the treatment progress collection apparatus 1 that collects data of rewards include a computer terminal, etc. used by a doctor during a medical diagnosis and treatment.

The treatment progress storage device 3 is a computer that includes a storage device for storing treatment progress data D (sₜⁱ, aₜ^{(i,j)}, sₜ₊₁ⁱ, rₜⁱ) relating to combinations of a patient i and a doctor j. As the storage device, a ROM (read only memory), a RAM (random access memory), an HDD (hard disk drive), an SSD (solid state drive), or an integrated circuit storage device, etc. storing various types of information may be used. The treatment progress data is managed for each combination of a patient i and a doctor j.

The medical learning apparatus 5 is a computer that generates an AI model (improved doctor model, a second inference model) that infers an optimal treatment action aₜ^{(i,j)} that should be taken by a doctor j for a state sₜⁱ of a patient i based on the treatment progress data relating to combinations of a patient i and a doctor j. Other than this AI model, the medical learning apparatus 5 may generate an AI model (doctor model, a first inference model) that infers a treatment action aₜ^{(i,j)} that a doctor j is expected to take for a state sₜⁱ of a patient i and an AI model (patient model, a third inference model) that infers a state sₜ₊₁ⁱ of a patient i that may emerge when a treatment action aₜ^{(i,j)} is given to a patient i who is in the state sₜⁱ. A doctor model is generated for each doctor j, an improved doctor model for each combination of a doctor j and a patient i, and a patient model for each patient i.

The AI model storage device 7 is a computer that includes a storage device storing a doctor model, an improved model, and a patient model generated by the medical learning apparatus 5. As the storage device, a ROM, a RAM, an HDD, an SSD, or an integrated circuit storage device may be used. A doctor model is stored for each doctor j, an improved doctor model for each combination of a doctor j and a patient i, and a patient model for each patient i.

The medical inference apparatus 9 is a computer that infers optimal treatment action aₜ^{(i,j)} that should be taken by a doctor j for a state sₜⁱ of a patient i, using an improved doctor model.

### First Embodiment

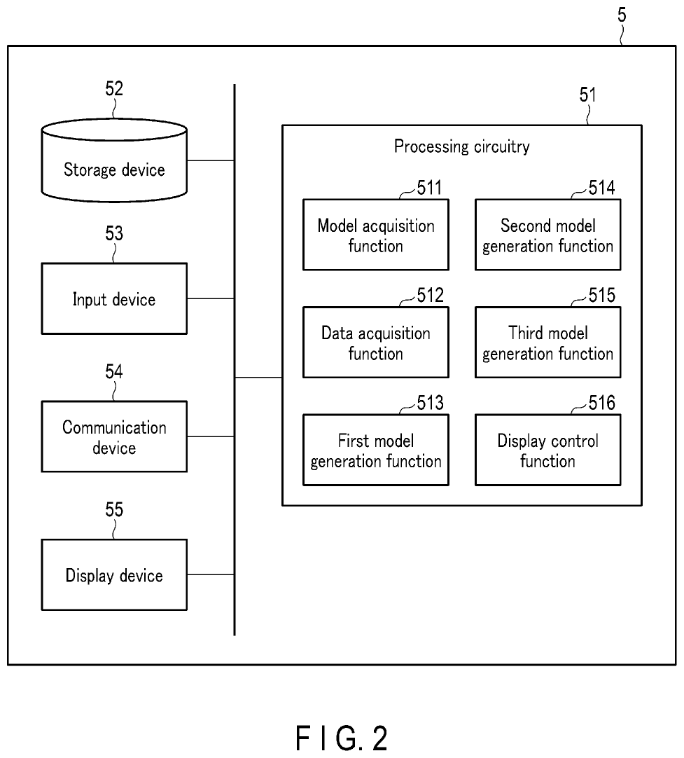

FIG. 2 is a diagram showing a configuration example of the medical learning apparatus 5 according to the first embodiment. As shown in FIG. 2, the medical learning apparatus 5 is an information processing terminal, such as a computer having processing circuitry 51, a storage device 52, an input device 53, a communication device 54, and a display device 55. The processing circuitry 51, the storage device 52, the input device 53, the communication device 54, and the display device 55 are connected to each other via a bus in such a manner that a communication can be mutually conducted.

The processing circuitry 51 includes processors such as a CPU (central processing unit) and a GPU (graphics processing unit). The processing circuitry 51 executes a medical learning program to realize a model acquisition function 511, a data acquisition function 512, a first model generation function 513, a second model generation function 514, a third model generation function 515, and a display control function 516. Note that the embodiment is not limited to the case in which the respective functions 511 to 516 are realized by single processing circuitry. Processing circuitry may be composed by combining a plurality of independent processors, and the respective processors may execute programs, thereby realizing the functions 511 to 516. The functions 511 and 516 may be respective modularized programs constituting a medical learning program. These programs are stored in the storage device 52.

The storage device 52 is a ROM (read only memory), a RAM (random access memory), an HDD (hard disk drive), an SSD (solid state drive), or an integrated circuit storage device, etc. storing various types of information. The storage device 52 may not only be the above-listed memory apparatuses but also a driver that writes and reads various types of information in and from, for example, a portable storage medium such as a compact disc (CD), a digital versatile disc (DVD), or a flash memory, or a semiconductor memory. The storage device 52 may be provided in another computer connected via a network.

The input device 53 accepts various kinds of input operations from an operator, converts the accepted input operations to electric signals, and outputs the electric signals to the processing circuitry 51. Specifically, the input device 53 is connected to an input device, such as a mouse, a keyboard, a trackball, a switch, a button, a joystick, a touch pad, or a touch panel display. The input device 53 outputs electrical signals to the processing circuitry 51 according to an input operation. An audio input apparatus may be used as an input device 53. The input device 53 may be an input device provided in an external computer connected to the system via a network, etc.

The communication device 54 is an interface for sending and receiving various types of information to and from other computers. An information communication by the communication device 54 is performed in accordance with a standard suitable for medical information communication, such as DICOM (digital imaging and communications in medicine).

The display device 55 displays various types of information in accordance with the display control function 115 of the processing circuitry 51. For the display device 55, for example, a liquid crystal display (LCD), a cathode ray tube (CRT) display, an organic electro luminescence display (OELD), a plasma display, or any other display can be used as appropriate. A projector may be used as the display device 55.

Through realization of the model acquisition function 511, the processing circuitry 51 acquires a doctor model (first inference model) that infers a treatment action of a target medical care provider (target doctor) based on a state of a patient. As an example, the processing circuitry 51 acquires a doctor model from the AI model storage device 7 via the communication device 54. A “target doctor” denotes a doctor for whom an improved doctor model is generated. A “target doctor” may be a specific individual or a statistically average doctor in a specific group. A “patient” includes not only a patient targeted before the customization of an improved doctor model generated based on a doctor model (namely, a “target patient”) but also other patients. The processing circuitry 51 may acquire an improved doctor model (second inference model) and a patient model (third inference model).

Through the realization of the data acquisition function 512, the processing circuitry 51 acquires treatment action progress data relating to a patient. The “treatment progress data relating to a patient” includes a state of a patient at a certain time point, a doctor's treatment action taken for the patient who is in the state, a state of the patient at a next time point after receiving the treatment action, and a reward for the treatment action. In other words, a doctor as an agent of the treatment action is not particularly limited. The “patient” may be a specific individual or a statistically average patient of a specific group. The processing circuitry 51 acquires treatment progress data relating to a patient from the treatment progress storage device 3 via the communication device 54.

Through realization of the first model generation function 513, the processing circuitry 51 generates a doctor model, which is an AI model that imitates a target doctor's decision-making for a patient based on treatment progress data relating to the target doctor. The “treatment progress data relating to a patient” includes a state of a patient at a certain time point, a target doctor's treatment action taken for the patient who is in the state, and a state of the patient at a next time point after the treatment action is received. In other words, the doctor as an agent of a treatment action is limited to a target doctor. The “treatment progress data” means treatment progress data relating to a combination of a target doctor and a patient. Specifically, a state of the patient at a certain time point is input to the doctor model, and the doctor model outputs a treatment action that the target doctor is expected to take for the patient who is in the state. The doctor model is there only to imitate a target doctor's decision-making relating to a treatment action, and does not concern itself with decision-making rationality. The generated doctor model is stored in the AI model storage device 7, being associated with an identifier of a target doctor corresponding to the doctor model. A reward may be included in the “treatment progress data” as needed.

Through realization of the second model generation function 514, the processing circuitry 51 generates, by updating the doctor model, an improved doctor model in conformity with a target patient (second inference model) based on the treatment progress data relating to the target patient. It suffices that the treatment progress data used in the generation of an improved doctor model be treatment progress data relating to the target patient. In other words, the doctor as an agent of the treatment action included in the treatment progress data is not limited to a target doctor and may be any doctor. As an improved doctor model is generated by updating a doctor model that is personalized for each individual doctor, initial values differ between such models even if they are generated using the same treatment progress data. It is therefore possible to generate an improved doctor model unique to each doctor (target doctor).

The improved doctor model is an AI model that infers the optimal decision-making of a target doctor for a target patient. A state of a target patient at a certain time point is input to the improved doctor model, and the model outputs a treatment action that should be taken by a target doctor for the target patient who is in this state. Suppose at least one of a “target patient” or a “target doctor” is a specific individual. The generated improved doctor model is stored in the AI model storage device 7, being associated with an identifier of a target doctor corresponding to the improved doctor model and an identifier of a target patient. A reward is not an essential element of the “treatment progress data relating to a target patient”.

Through realization of the third model generation function 515, the processing circuitry 51 generates a patient model (third inference model) that infers, based on treatment progress data relating to a target patient, a state which the target patient may be in at a next time point following a certain treatment action given to the target patient who was in a certain state at a certain time point. Specifically, a state of a target patient at a certain time point and a treatment action given to the target patient who is in this state are input to the patient model, which then outputs a state that the target patient may be in at a next time point. The generated patient's model is stored in the AI model storage device 7, being associated with an identifier of a target patient corresponding to the patient model.

Through realization of the display control function 115, the processing circuitry 51 causes the display device 55 to display various information items. As an example, the processing circuitry 51 triggers learning results, etc. by the doctor model, the improved doctor model, and the patient model to be displayed.

Hereinafter, the medical learning processing by the medical learning apparatus 500 according to the first embodiment is described.

### Example 1

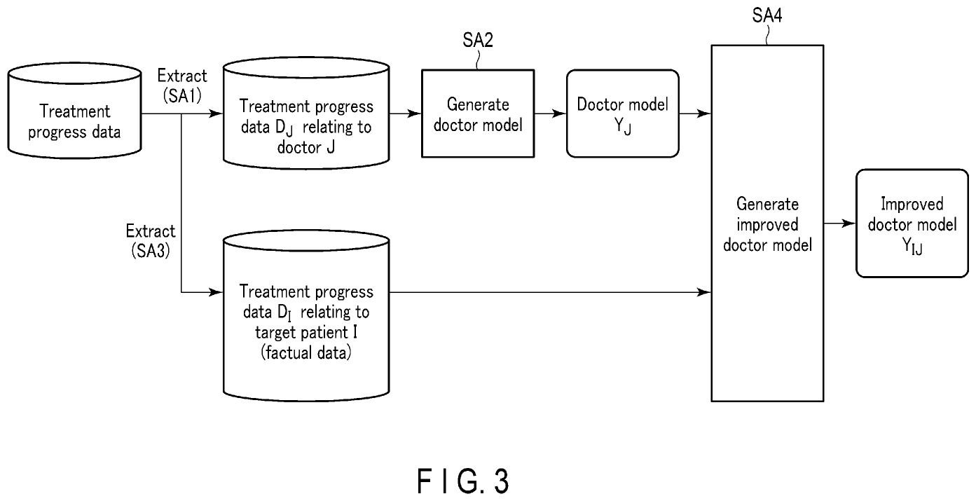

FIG. 3 is a diagram schematically showing a processing sequence for medical learning processing by the medical learning apparatus 500 according to Example 1 of the first embodiment. Assume that treatment progress data relating to various combinations of a doctor and a patient is stored in the treatment progress storage device 3 at a time of starting the medical learning processing.

As shown in FIG. 3, the processing circuitry 51 extracts, through realization of the data acquisition function 512, treatment progress data D_{J}(sₜⁱ, aₜ^{(i,J)}, sₜ₊₁ⁱ, rₜⁱ) relating to a target doctor J from the treatment progress data D stored in the treatment progress storage device 3 (step SA1). Treatment progress data D_{J} relating to a target doctor J is treatment progress data relating to combinations of interactions between the target doctor J and various patients i. Herein, the patients i include not only a target patient I but also any discretionarily selected patients. The treatment progress data D_{J} relating to the target doctor J is factual data, in other words, actually measured data.

After step SA1, the processing circuitry 51 generates, through realization of the first model generation function 513, a doctor model Y_{J} relating to the target doctor J based on the treatment progress data D_{J} relating to the target doctor J (step SA2). The doctor model Y_{J} is a policy model that imitates target doctor J's decision-making relating to a treatment action. The doctor model Y_{J} is generated based on treatment action data of at least the target doctor J. The treatment action data includes data of a treatment action taken by the target doctor J for a predetermined state of the patient i.

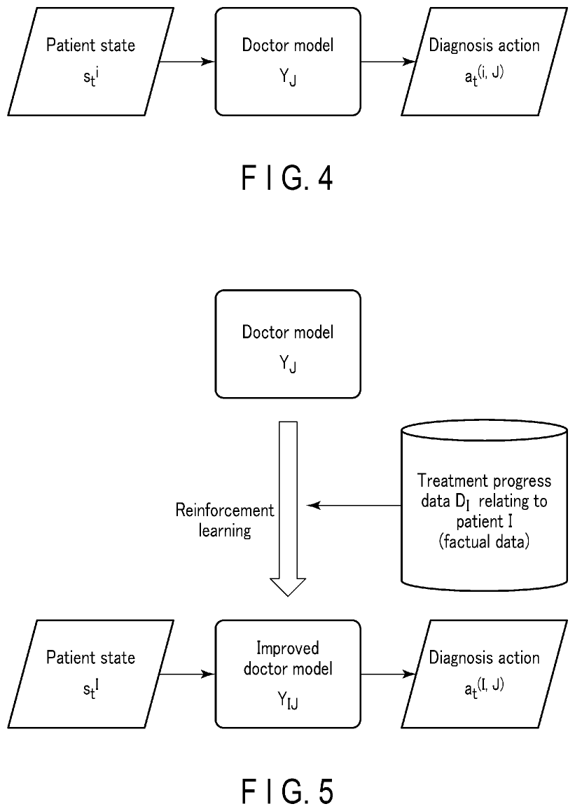

FIG. 4 is a diagram showing an input and an output of the doctor model Y_{J}. As shown in FIG. 4, a state sₜⁱ of the patient i at a time point t is input to the doctor model Y_{J}, and the doctor model Y_{J} outputs a treatment action aₜ^{(i,J)} that the doctor J is expected to take for the patient i. The state sₜⁱ has a form of a vector having predetermined multiple types of state elements. The treatment action aₜ^{(i,J)} is acquired by an output format of a multi-class classification. In other words, the treatment action aₜ^{(i,J)} has a vector format having predetermined multiple types of treatment action elements.

There are various methods for generating a doctor model YJ. As an example, the processing circuitry 51 generates a doctor model Y_{J}(aₜ^{(i,J)}, sₜⁱ) by training a policy model through behavior cloning or imitation learning based on a state sₜⁱ and a treatment action aₜ^{(i,J)}. Herein, imitation learning includes apprenticeship learning in which reinforcement learning and inverse reinforcement learning are combined. As imitation learning, GAIL (generative adversarial imitation learning) may be adopted. The processing circuitry 51 may train the doctor model Y_{J} with an input of a time-invariant feature amount in addition to an input of a state sₜⁱ. The time-invariant feature amount means a feature amount of a doctor and/or a patient that does not vary with time, for example sex, blood type, clinical department, nationality, race, etc. It is expected that adding a time-invariant feature amount to an input improves accuracy in the output of treatment action aₜ^{(i,J)}.

As shown in FIG. 3, the processing circuitry 51 extracts, through realization of the data acquisition function 512, treatment progress data DI relating to the target patient I from the treatment progress data D stored in the treatment progress storage device 3 (step SA3). Treatment progress data D_{I} relating to a target patient I is treatment progress data relating to combinations of interactions between the target patient I and various doctor j. A “doctor j” herein means not only a “target” doctor J for whom an improved doctor model is generated but also other discretionarily selected doctors. The treatment progress data D relating to the target patient I is factual data, in other words, actually measured data.

After steps SA2 and SA3, the processing circuitry 51 generates, through realization of the second model generation function 514, an improved doctor model Y_{IJ}, which is a doctor model of a target doctor J in conformity with the target patient I, through reinforcement learning based on treatment progress data DI (factual data) relating to the target patient I (step SA4). As reinforcement learning, on-policy learning such as TRPO (Trust Region Policy Optimization) and PPO (Proximal Policy Optimization), and off-policy learning such as DQN (Deep Q-Networks) and SAC (Soft-Actor-Critic) may be adopted.

FIG. 5 is a diagram showing an input and an output of the improved doctor model Y_{IJ}. As shown in FIG. 5, the improved doctor model Y_{IJ} is an AI model that infers an optimal treatment action aₜ^{(I,J)} taken by the target doctor J for the target patient I. A state s of the target patient I at a time point t is input to the improved doctor model Y_{IJ}, which then outputs an optimal treatment action aₜ^{(I,J)} that the doctor J should take for the target patient I.

There are various methods for generating an improved doctor model Y_{IJ}. As an example, the processing circuitry 51 generates an improved doctor model by training a policy model having a doctor model Y_{J} as an initial value through reinforcement learning based on the treatment progress data D_{I} relating to the target patient I. As the treatment progress data DI, a state sₜⁱ of the target patient I, a treatment action aₜ^{(I,j)}, and a reward rₜ^{I} are used. As an example, learning parameters (weight parameters, bias) of the doctor model Y_{J} are updated using a Q value (action value) corresponding to a predicted treatment action that is output by the doctor model Y_{J} based on a state sₜ^{I}, so that an objective function of a policy gradient method becomes maximum. Until a condition for finishing the updates is satisfied, the learning parameters are repeatedly updated. If the condition for finishing the updates is satisfied, the training of the improved doctor model Y_{IJ} ends.

Some constraint on the degree of change from the initial value of the policy model may be introduced to ensure that the difference between the improved physician model Y_{IJ} and the physician model Y_{J} is not too large. For example, a technique can be used to update the policy function with a KL distance constraint between the policies in the policy model before and after the update.

The medical learning process according to Example 1 is thus finished.

### Example 2

The medical learning apparatus 5 according to Example 2 generates an improved doctor model using counterfactual treatment progress data generated based on a patient model.

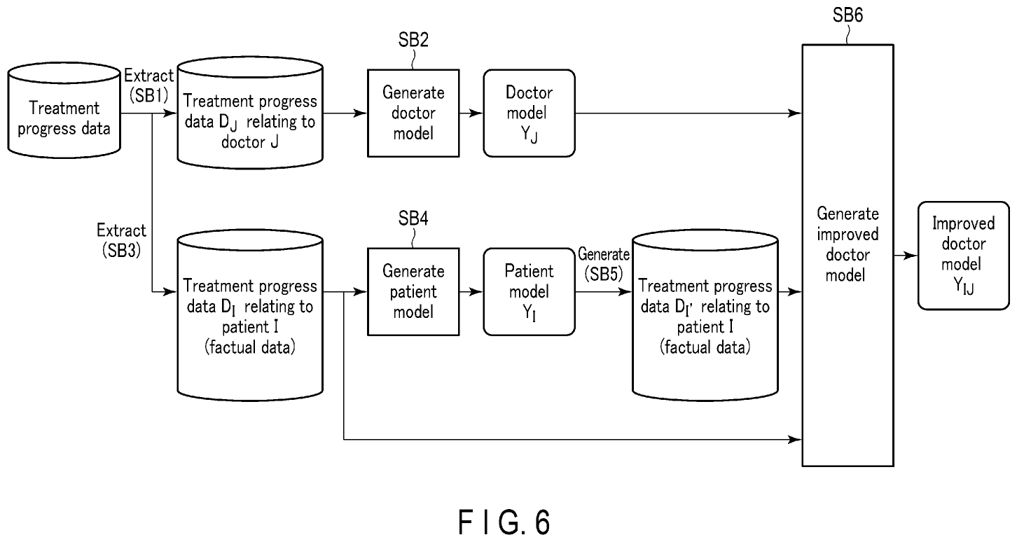

FIG. 6 is a diagram schematically showing a processing sequence for medical learning processing by the medical learning apparatus 500 according to Example 2 of the first embodiment. Assume that treatment progress data relating to various combinations of a doctor and a patient is stored in the treatment progress storage device 3 at a time of starting medical learning processing. Since steps SB1 to steps SB3 are the same as steps SA1 to SA3 in FIG. 3, the description of steps SB1 to steps SB3 is omitted.

After step SB3, the processing circuitry 51 generates, through realization of the third model generation function 515, a patient model Y_{I} relating to the target patient I based on the treatment progress data D_{I} relating to the target patient I (step SB4). The patient model Y_{I} outputs a state sₜ₊₁^{I} of the target patient I at a next time point t+1 following a treatment action aₜ^{(I,j)} given to the target patient I in the state of sₜ^{I} at a time point t.

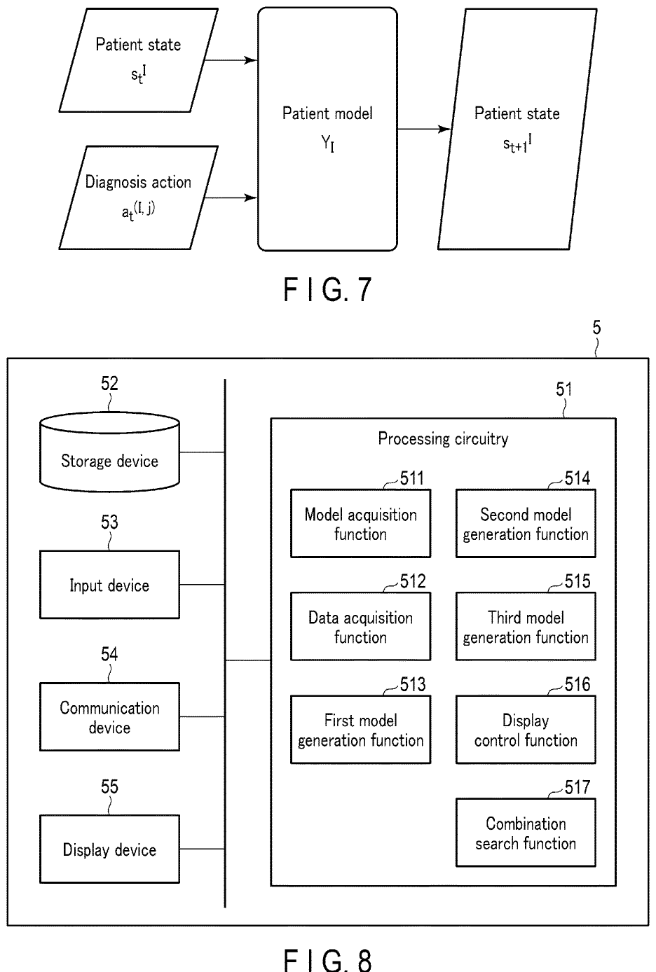

FIG. 7 is a diagram showing an input and an output of the patient model Y_{I}. As shown in FIG. 7, the patient model YI outputs a state sₜ₊₁^{I} of the target patient I at a next time point t+1 based on the state of sₜⁱ of the target patient I at a time point t and the treatment action aₜ^{(I,j)} given to the target patient I by the doctor j. The state sₜ₊₁^{I} is acquired by an output format of a multi-class classification. In other words, the state sₜ₊₁^{I} can be acquired by a vector format having multiple predetermined types of state elements.

There are various methods for generating a patient model Y_{I}. As an example, the processing circuitry 51 generates a patient model Y_{I} by training an environment model T_{I}(sₜ₊₁^{I}|sₜ^{I}, aₜ^{(I,j)}) or T_{I}(sₜ₊₁^{I}, rₜ^{I}|sₜ^{I}, aₜ^{(I,j)}) based on the treatment progress data D_{I} relating to the target patient I. As another example, the processing circuitry 51 may generate a patient model Y_{I} by ensemble learning. Specifically, the processing circuitry 51 first generates a plurality of environment models T_{I}(sₜ₊₁^{I}|sₜ^{I}, aₜ^{(I,j)}) or T_{I}(sₜ₊₁^{I}, rₜ^{I}|sₜ^{I}, aₜ^{(I,j)}) relating to the target patient I. The plurality of environment models T_{I} may be generated by setting initial values of hyper parameters and learning parameters (weight parameters and bias) to random numbers generated by a random number generator and training the untrained environment models T_{I} with a chronological prediction task, etc. The processing circuitry 51 forms a linearly connected network of the plurality of environment models T_{I}. The weight parameters of the linearly connected network may be determined by machine learning. The patient model YI is thus generated.

As another example, the processing circuitry 51 may generate a patient model YI in consideration of a causal structure between a combination of a state s and a treatment action aₜ^{(I,j)} and a state sₜ₊₁^{I}, or may use, as a patient model YI, a simulation model in which the relationship between a combination of a state sₜ^{I} and a treatment action aₜ^{(I,j)} and a state sₜ₊₁^{I} is expressed in a mathematical expression as preliminary knowledge. The processing circuitry 51 may generate a patient model Y_{I} using a continuous time (Neural ODE).

After step SB4, the processing circuitry 51 generates, through realization of the data acquisition function 512, counterfactual treatment progress data D_{I}′ relating to the target patient I based on the patient model Y_{I} (step SB5). Specifically, at step SB5, the processing circuitry 51 first generates a treatment action aₜ^{(I,j)′} by applying a state sₜ^{I′} which is a target of generation for the improved doctor model YIJ. The state sₜ^{I′} is not necessarily actually measured factual data and may be counterfactual data generated artificially or by a random number generator. The treatment action aₜ^{(I,j)′} is counterfactual data that is not actually measured. The processing circuitry 51 generates a state sₜ₊₁^{I′} by applying the state sₜ^{I′} and the treatment action aₜ^{(I,j)′} to the patient model Y_{I}. The reward rₜ^{I′} may be calculated by applying the treatment action aₜ^{(I,j)′} to a discretionarily selected reward function. A combination of the state sₜ^{I′}, the treatment action aₜ^{(I,j)′}, the state sₜ₊₁^{I′}, and the reward rₜ^{I′} constitutes one sample of counterfactual treatment progress data. A plurality of samples of the counterfactual treatment progress data are generated by recursively performing the above-described series of processes with the change of the time t of the state sₜ^{I′}.

After step SB2 and step SB5, the processing circuitry 51 generates, through realization of the second model generation function 514, an improved doctor model Y_{IJ} by reinforcement learning based on treatment progress data (factual data) D relating to a target patient I and treatment progress data (counterfactual data) D_{I}′ (step SB6). At step SB6, unlike step SA4 in Example 1, the treatment progress data (counterfactual data) D_{I} is used in addition to the treatment progress data (factual data) D_{I}′. Other than for this point, the process in step SB6 is the same as step SA4. Since using the treatment progress data (counterfactual data) D_{I}′ increases the diversity of data used in reinforcement learning, the accuracy of the improved doctor model Y_{IJ} is expected to improve. The improved doctor model Y_{IJ} may be generated based on either one of the treatment progress data (counterfactual data) D_{I} or the treatment progress data (factual data) D_{I}′.

The medical learning process according to Example 2 is thus finished.

The processing circuitry 51 can update an improved doctor model through realization of the second model generation function 514. As an example, the processing circuitry 51 updates an improved doctor mode based on new treatment progress data D_{I} and/or D_{I}′ relating to a time point later than a previously determined time point. It suffices that the updating process be performed at regular intervals or prompted by an instruction of an operator at a discretional timing. Preferably, the treatment progress data D_{I} and/or D_{I}′ used in the updating process includes only new treatment progress data D_{I} and/or D_{I}′ that was not used in a previous updating process. By performing the updating process using only the new treatment progress data D_{I} and/or D_{I}′, it is possible to exclude past insights and adopt the latest insights into the improved doctor model; therefore, the accuracy of the output of the improved doctor model is expected to improve. Since the accuracy of the treatment progress data D_{I}′ is expected to improve every time it is repeatedly generated, the past treatment progress data D_{I}′ can be discarded, the updating process can be therefore performed using only the new treatment progress data D_{I}′, and the accuracy of the output of the improved doctor model is thus expected to improve. The treatment progress data D_{I} and/or D_{I}′ used in the updating process before the one immediately prior may be used to update the improved doctor model.

As described above, the medical learning system 100 includes the processing circuitry 51. The processing circuitry 51 acquires a first inference model that infers a treatment action of a target doctor based on a state of a patient. The processing circuitry 51 acquires treatment progress data of the target patient. The processing circuitry 51 generates an improved doctor model (second inference model) in conformity with the target patient by updating a doctor model (first inference model) based on the treatment progress data.

According to the above configuration, an improved doctor model of a target doctor that is an inference model customized for a target patient can be generated. The improved doctor model is customized for a medical diagnosis and treatment policy of a target doctor for a target patient in contrast to an inference model trained based on treatment progress data relating to a plurality of combinations of doctor-patient combinations. Therefore, it is possible to realize CDS in which individual doctor expertise and senses of values are exploited. Consequently, each patient can receive optimal medical diagnoses and treatment actions from a doctor. As an improved doctor model is an improved version of a doctor model that imitates a target doctor, it is expected that its policy is near to that of the target doctor; therefore, it is possible for the target doctor to easily accept a treatment action inferred by the improved doctor model. Furthermore, it is possible for the target doctor to acquire new knowledge by comparing their own intended medical diagnoses and treatment actions with those inferred by the improved doctor model.

### Second Embodiment

The medical learning apparatus 5 according to the second embodiment searches among a plurality of patient models corresponding to a plurality of patients and a plurality of doctor models corresponding to a plurality of doctors for an optimal combination. Hereinafter, the medical learning system according to the second embodiment will be described below.

FIG. 8 is a diagram showing a configuration example of the medical learning apparatus 5 according to the second embodiment. As shown in FIG. 8, the processing circuitry 51 of the medical learning apparatus 5 realizes a combination search function 517 in addition to the model acquisition function 511, the data acquisition function 512, the first model generation function 513, the second model generation function 514, the third model generation function 515, and the display control function 516. Through realization of the combination search function 517, the processing circuitry 51 searches among a plurality of doctor models corresponding to a plurality of doctors and a plurality of patient models corresponding to a plurality of patients for an optimal combination. The processing circuitry 51 generates an improved doctor model by updating the doctor model belonging to the optimal combination based on treatment progress data relating to a target patient corresponding to the patient model belonging to the optimal combination.

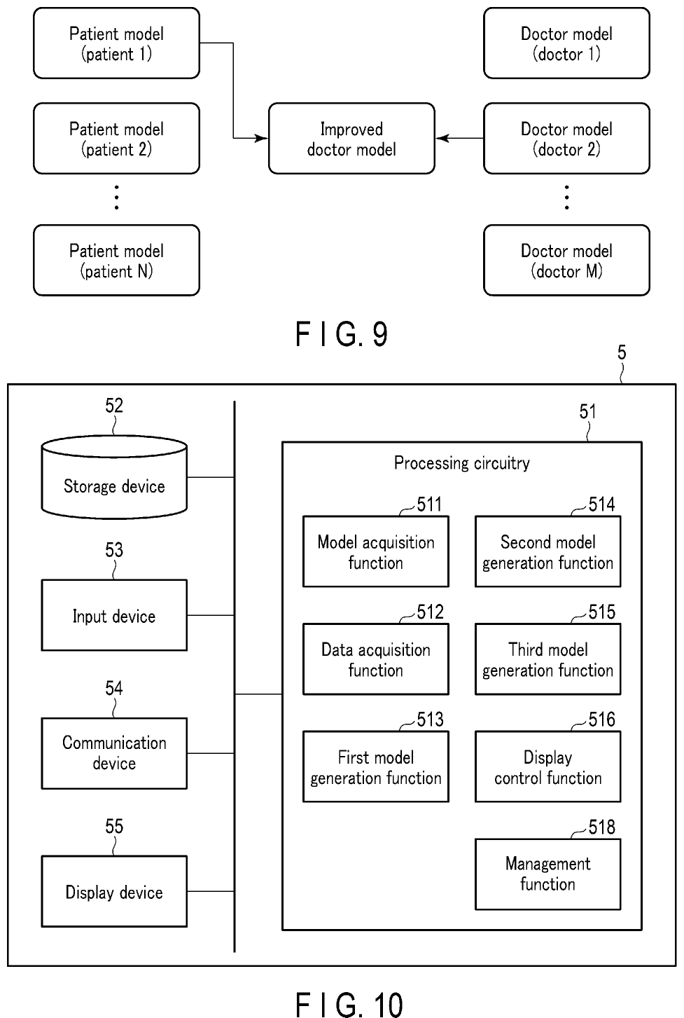

FIG. 9 is a diagram schematically showing an example of a search process in the second embodiment. As shown in FIG. 9, suppose N patient models respectively corresponding to N patients and M doctor models respectively corresponding to M doctors are prepared (N and M are a natural number). The processing circuitry 51 searches N patient models and M doctor models for an optimal combination.

Herein, M doctor models are searched for a doctor model corresponding to a doctor who is optimal for a patient model of a patient 1. The processing circuitry 51 compares the performance of the improved doctor model acquired from each of the M doctor models with the fixed patient model for the patient 1, and sets the doctor model corresponding to an improved doctor model having the best performance to an optimal combination for the patient model the patient 1.

Specifically, the doctor model of the doctor 2 is updated by reinforcement learning based on the factual treatment progress data relating to the patient 1 and the counterfactual treatment progress data relating to the patient 1, and the improved doctor model is thereby generated. Next, the processing circuitry 51 calculates an index (performance evaluation index) for evaluating performance of the generated improved doctor model. The performance evaluation index is not limited to any specific index, and for example, a stratified boot strap confidence interval, performance profile, quartile mean, and improved probability may be used. The processing circuitry 51 generates an improved doctor model for other doctor models in a similar manner, and calculates a performance evaluation index for the generated improved doctor model. The generation of an improved doctor model and calculation of a performance evaluation index may be performed for all M doctor models or for randomly selected doctor models. Then, the processing circuitry 51 selects a doctor model corresponding to an improved doctor model having a highest performance evaluation index value as an optimal doctor model for the patient model of the patient 1. In the case shown in FIG. 9, a combination of the patient model of the patient 1 and the doctor model of the doctor 2 is an optimal combination.

As another example, the processing circuitry 51 may search a plurality of patient models for a patient model corresponding to a patient optimal for a specific doctor. In this case, the processing circuitry 51 generates an improved doctor model based on the patient model and the specific doctor's doctor model for each of the patient models respectively corresponding to a plurality of patients. Specifically, a doctor model of a specific doctor is updated by reinforcement learning based on factual treatment progress data relating to a patient, and counterfactual treatment progress data relating to the patient obtained based on a patient's patient model, thereby generating an improved doctor model. Next, the processing circuitry 51 calculates a performance evaluation index of the generated improved doctor model. The processing circuitry 51 generates an improved doctor model for other patient models in a similar manner, and calculates a performance evaluation index for the generated improved doctor model. The generation of an improved doctor model and calculation of a performance evaluation index may be performed for all N patient models or randomly selected patient models. Then, the processing circuitry 51 selects a patient model corresponding to an improved doctor model having the highest performance evaluation index value as an optimal patient model for the doctor model of a specific doctor.

As another example, the processing circuitry 51 may search for an optimal doctor model for a patient model of a specific patient based on Bayes optimization in which a feature amount of a doctor model is used as a parameter. As the feature amount, a feature amount relating to a doctor, such as a doctor's age or practice area, and a feature amount of a doctor model, such as the number of the layers of the doctor model, may be used.

The processing circuitry 51 may perform reinforcement learning on a combination of a plurality of doctor models to generate a single improved doctor model relating to the plurality of doctors. A method of integrating improved doctor models may be performed through majority selection of an action, probabilistic selection of an action, or averaging parameters. An integration ratio may be changed discretionarily.

According to the second embodiment, it is possible to search for an optimal combination of a patient model and a doctor model, and it is therefore possible to generate an improved doctor model optimal for a specific patient or a specific doctor.

### Third Embodiment

The medical learning system 100 according to the third embodiment manages an improved doctor model in a distributed database such as a block chain. Hereinafter, the medical information processing system according to the third embodiment will be described below.

FIG. 10 is a diagram showing a configuration of the medical learning apparatus 5 according to the third embodiment. As shown in FIG. 8, the processing circuitry 51 of the medical learning apparatus 5 realizes a management function 518 in addition to the model acquisition function 511, the data acquisition function 512, the first model generation function 513, the second model generation function 514, the third model generation function 515, and the display control function 516. Through realization of the management function 518, the processing circuitry 51 manages an improved doctor model in a block chain. A block chain is a series of blocks partially recording a history of updates and inference of an improved doctor model, and a history of treatment progress data used in the updating and inference. At the time of inference using an improved doctor model, the processing circuitry 51 adds an improved doctor model and treatment progress data used in the inference to a block, associating the model and the data with each other. At the time of updating an improved doctor model, the processing circuitry 51 adds an improved doctor model and treatment progress data used in the inference to a block, associating them with each other. The processing circuitry 51 can update, through realization of the second model generation function 514, an improved doctor model based on treatment progress data relating to a time point that comes later than a time point related to treatment progress data used in the generation of the improved doctor model.

It is not sufficient to implement the management function 518 in a specific medical learning apparatus 5 included in the medical learning system 100, and suppose that the management function 518 is also implemented in other computers, such as the treatment progress collection apparatus 1, the treatment progress storage device 3, the medical learning apparatus 5, the AI model storage device 7, and/or the medical inference apparatus 9. The improved doctor model in the third embodiment is stored in a block chain, and is not necessarily stored in the AI model storage device 7.

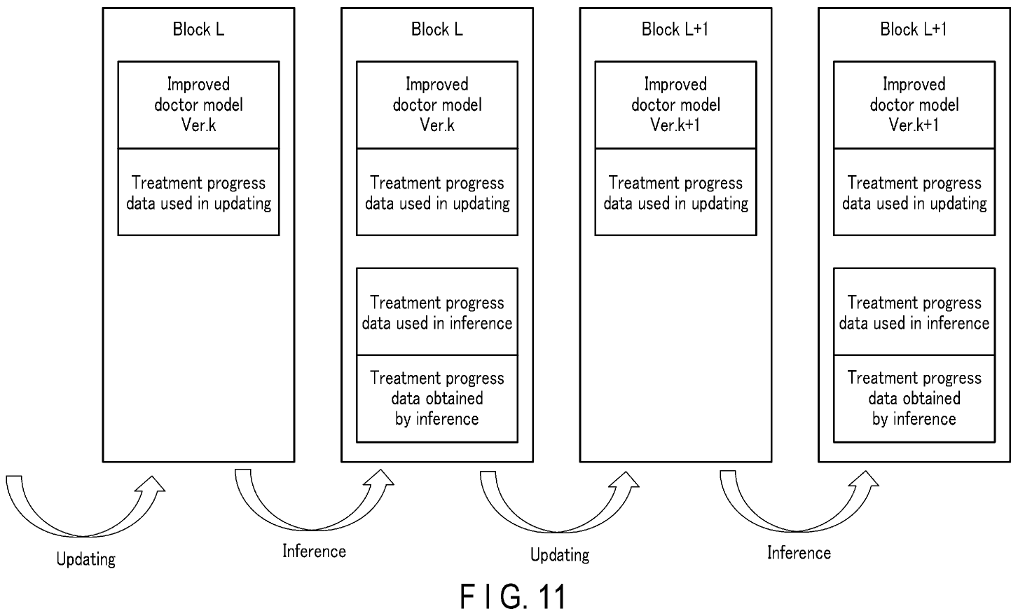

FIG. 11 is a drawing schematically showing a management process of an improved doctor model. As shown in FIG. 11, the block chain managing an improved doctor model includes a series of L (natural number) blocks, and the transition of the latest block is illustrated. An improved doctor model and treatment progress data are stored in each block. A hash value in which a transaction in a previous block and Nonce, a parameter obtained through hash calculation, are stored in each block; however, the illustration of these values is omitted.

As shown in FIG. 11, if an improved doctor model is updated to version k through the second model generation function 514, the processing circuitry 51 causes, through the management function 518, an improved doctor model of version k, as well as treatment progress data used in the updating to be stored in the latest L-th block. In a block, both the data and an identifier for an improved doctor model may be stored. When an identifier is stored, the data for an improved doctor model corresponding to the identifier is stored in the AI model storage device 7, being associated with the identifier.

As shown in FIG. 11, if inference is conducted by the medical inference apparatus 9 using an improved doctor model of version k, the processing circuitry 51 adds, through the realization of the management function 518, the treatment progress data used in the inference and treatment progress data obtained through the inference to L-th block. Only one of the treatment progress data used in the inference or the treatment progress data obtained through the inference may be added to L-th block.

As shown in FIG. 11, if an improved doctor model is updated from version k to version k+1 through the second model generation function 514, the processing circuitry 51 causes, through the management function 518, an improved doctor model of version k+1 and treatment progress data used in the updating to be stored in the latest (L+1)-th block. Thereafter, if inference is conducted by the medical inference apparatus 9 using an improved doctor model of version k+1, the processing circuitry 51 adds, through the realization of the management function 518, the treatment progress data used in the inference and treatment progress data obtained through the inference to (L+1)-th block.

In FIG. 11, the improved doctor model obtained through updating is stored in a new block; however, the present embodiment is not limited to this example, and the improved doctor model and the treatment progress data used in the updating of the improved doctor model may be stored in a new block every certain period of time. In this case, a plurality of improved doctor models and/or a plurality of treatment progress data sets are stored in a single block. The improved doctor model used in the inference is stored in a current block; however, the present embodiment is not limited to the example. If the model cannot be stored in a current block, it may be stored in a new block.

As stated above, according to the third embodiment, since an improved doctor model and treatment progress data are stored in a block chain, in contrast to the case where the model and data are stored in the AI model storage device, it is possible to reduce the risk of tampering. Since a model is stored at the timing of the performance of updates or inference or every certain period of time, it is possible to ensure that an improved doctor model and treatment progress data are stored in a block chain.

### Development Examples

A doctor model according to an development example is a multi-head inference model having M output layers corresponding to M doctors. Similarly, a patient model according to the applied model is a multi-head inference model having N output layers corresponding to N patients.

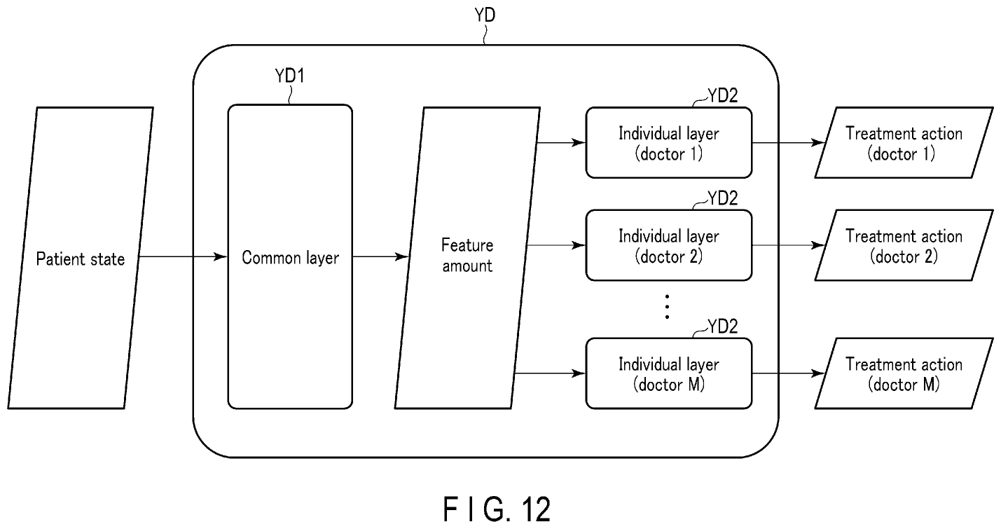

FIG. 12 is a diagram showing an example of a network architecture of the doctor model YD according to the development example. As shown in FIG. 12, the doctor model YD has a common layer YD1 and M individual layers YD2. The common layer YD1 is a network layer that is common between M doctors. A state sₜⁱ of a patient i at a time point t is input to the common layer YD1 and an intermediate output is output therefrom. An intermediate output is a vector quantity of a lower dimension in contrast to the state sₜⁱ. M individual layers YD2 are network layers respectively corresponding to M doctors. Each of M individual layers YD2 to which an intermediate output is input thus outputs a treatment action aₜ^{(i,j)} of a doctor j corresponding to the patient i.

The processing circuitry 51 generates a doctor model YD through realization of the first model generation function 513. As an example, the processing circuitry 51 generates a doctor model YD through multi-task learning in which treatment action aₜ^{(i,j)} for M doctors is inferred. As another example, the processing circuitry 51 forms a network architecture of the doctor j by connecting a single individual layer YD2 to the common layer YD1 and generates a doctor model of the doctor j by training the network architecture through behavior cloning or imitation learning in the method similar to that in the first embodiment. Thereafter, a doctor model of other doctors may be subsequently generated through transfer learning based on the doctor model of the doctor j.

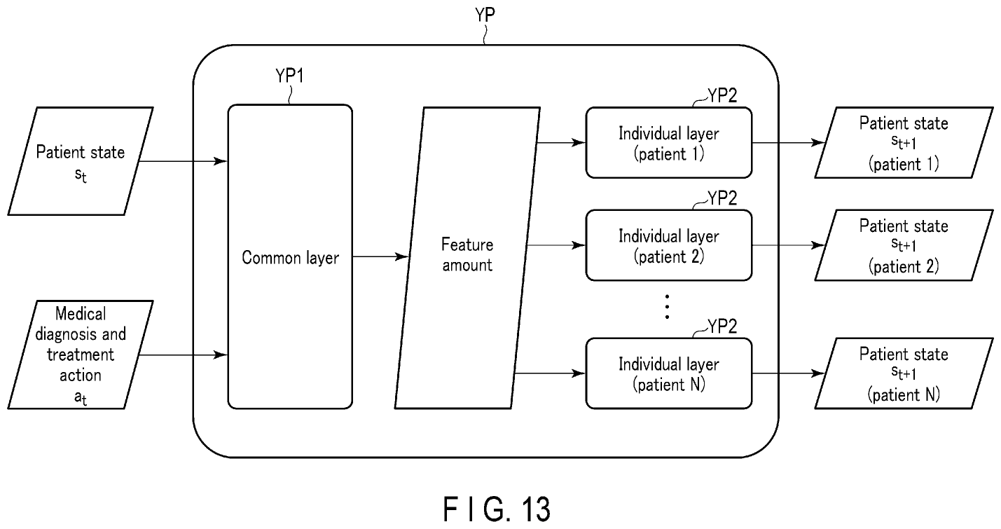

FIG. 13 is a diagram showing an example of a network architecture of a patient model YP according to the development example. As shown in FIG. 13, the patient model YP has a common layer YP1 and N individual layers YP2. The common layer YP1 is a network layer common between N patients. The common layer YP1 to which the state sₜⁱ of the patient i at a time point t and treatment action aₜ^{(i,j)} taken by the doctor j to the patient i are input thereby outputs an intermediate output. An intermediate output is a vector quantity of a low dimension in contrast to the state sₜⁱ and the treatment action aₜ^{(i,j)}. N individual layers YP2 is a network layer corresponding to N patients. Each of the N individual layers YP2, to which the intermediate output is input, thereby outputs a state sₜ₊₁ⁱ of the patient i at a next time point t+1.

The processing circuitry 51 generates a patient model YP through the third model generation function 515. As an example, the processing circuitry 51 generates a patient model YP through multi-task learning in which a state sₜ₊₁ⁱ for N patients is inferred. As another example, the processing circuitry 51 forms a network architecture of the patient i by connecting a single individual layer YP2 to the common layer YP1, and then generates a patient model of the patient i by training the network architecture through time-series prediction task, etc. in the method similar to that in the first embodiment. Thereafter, patient models of other patients may be subsequently generated through transfer learning based on the patient model of the patient i.

Hereinafter, the search process by the combination search function 517 according to this development example will be described.

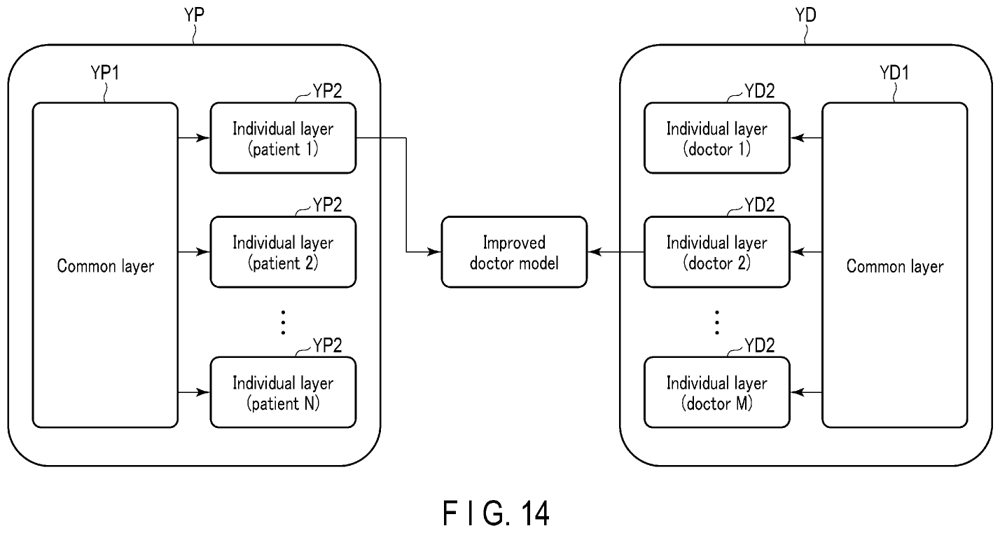

FIG. 14 is a diagram schematically showing a search process in the development example. As shown in FIG. 14, the patient model YP has a common layer YP1 and N individual layers YP2, and the doctor model YD has a common layer YD1 and M individual layers YD2. The processing circuitry 51 searches, through realization of the combination search function 517, N individual layers YP2 for an optimal individual layer YP2 for a specific individual layer YD2 among M individual layers YD2. The same method as that in the second embodiment may be adopted as a search method. Similarly, the processing circuitry 51 searches, through realization of the combination search function 517, M individual layers YD2 for an optimal individual layer YD2 for a specific individual layer YP2 among N individual layers YP2.

As an example, the processing circuitry 51 compares the performance of the improved doctor model obtained from each of M individual layers YD2 with the fixed individual layer YP2 of the patient 1, and sets the doctor model corresponding to an improved doctor model having the best performance as an optimal combination for the individual layer YP2 of the patient 1. Specifically, the individual layer YD2 of the doctor 2 is updated by reinforcement learning based on the factual treatment progress data relating to the patient 1 and the counterfactual treatment progress data relating to the patient 1 obtained based on the individual layer YP2 of the patient 1, and the improved doctor model is thereby generated. Next, the processing circuitry 51 calculates an index (performance evaluation index) for evaluating the performance of the generated improved doctor model. The processing circuitry 51 generates an improved doctor model for the individual layer YD2 of other doctor in a similar manner, and calculates a performance evaluation index for the generated improved doctor model. Then, the processing circuitry 51 selects an individual layer YD2 corresponding to an improved doctor model having the highest performance evaluation index value as an optimal individual layer YD2 for the individual layer YP2 of the patient 1. In the case shown in FIG. 9, a combination of the individual layer YP2 of the patient 1 and the individual layer YD2 of the doctor 2 is an optimal combination.

The doctor model and the patient model according to the foregoing development example is a multi-head inference model; however, an individual model of the doctor model and the patient model may be obtained through meta learning. As meta learning, model-agnostic meta-learning (MAML), neural process, prototype networks, and other methods may be adopted. Herein, “an individual model” refers to a network as a whole optimized for a doctor or a patient without having a multi-head architecture, akin to the doctor model shown in FIG. 4 and the patient model FIG. 7. MAML is a method of learning common good initial values at the time of learning an individual model. Since a network architecture between a plurality of individual models relating to a doctor model or to a patient model is common, it is possible to efficiently learn an individual model with a small quantity of data without having a multi-head architecture.

According to at least one of the foregoing embodiments, it is possible to achieve an AI model that can support a doctor's medical diagnosis and treatment taken for a patient with high accuracy, while each doctor's expertise and sense of values are exploited.

The term “processor” used in the above explanation indicates, for example, a circuit, such as a CPU, a GPU, or an Application Specific Integrated Circuit (ASIC), and a programmable logic device (for example, a Simple Programmable Logic Device (SPLD), a Complex Programmable Logic Device (CPLD), and a Field Programmable Gate Array (FPGA)). The processor realizes its function by reading and executing the program stored in the storage circuitry. The program may be directly incorporated into the circuit of the processor instead of being stored in the storage circuit. In this case, the processor implements the function by reading and executing the program incorporated into the circuit. If the processor is for example an ASIC, on the other hand, the function is directly implemented in a circuit of the processor as a logic circuit, instead of storing a program in a storage circuit. Each processor of the present embodiment is not limited to a case where each processor is configured as a single circuit; a plurality of independent circuits may be combined into one processor to realize the function of the processor. Further, a plurality of components shown in FIG. 1, FIG. 8 and FIG. 10 may be integrated into one processor to achieve their functions.

While certain embodiments have been described, these embodiments have been presented by way of example only, and are not intended to limit the scope of the inventions. Indeed, the embodiments described herein may be embodied in a variety of other forms; furthermore, various omissions, substitutions, changes, and combinations of embodiments in the form of the embodiment described herein may be made without departing from the spirit of the invention. The accompanying claims and their equivalents are intended to cover such forms or modifications as would fall within the scope and spirit of the invention.

What is claimed is:

1. A medical learning system comprising processing circuitry configured to:
acquire a first inference model that infers a treatment action of a target medical care provider based on a state of a patient;
acquire treatment progress data relating to a target patient; and
generate a second inference model by updating the first inference model based on the treatment progress data relating to the target patient.

2. The medical learning system of claim 1, wherein
the first inference model is generated based on the treatment action data of the target medical care provider.

3. The medical learning system of claim 2, wherein
the treatment action data includes data relating to a treatment action taken by the target medical care provider for a predetermined state of the patient.

4. The medical learning system of claim 2, wherein
the first inference model is a policy model to which the state is input and which outputs the treatment action, the policy model being generated through behavior cloning or imitation learning based on state data of the patient and the treatment action data of the target medical care provider.

5. The medical learning system of claim 1, wherein
the treatment progress data is actually measured data relating to the target patient.

6. The medical learning system of claim 1, wherein
the processing circuitry is further configured to:
acquire a third inference model that infers a treatment progress of the target patient; and
acquire data inferred by the third inference model as the treatment progress data.

7. The medical learning system of claim 1, wherein
the processing circuitry generates the second inference model by training a policy model using the first inference model as an initial value through reinforcement learning based on the treatment progress data.

8. The medical learning system of claim 7, wherein
the treatment progress data is factual data relating to the target patient.

9. The medical learning system of claim 7, wherein
the processing circuitry is further configured to:
acquire a third inference model that infers treatment progress of the target patient; and
acquire counterfactual data inferred by the third inference model as the treatment progress data.

10. The medical learning system of claim 1, wherein
the processing circuitry further searches among a plurality of first inference models respectively corresponding to a plurality of medical care providers and a plurality of third inference models respectively corresponding to a plurality of patients for an optimal combination.

11. The medical learning system of claim 1, wherein
the target medical care provider includes a plurality of medical care providers,
the first inference model includes a first common layer that is common between the plurality of medical care providers, and a plurality of first individual layers respectively corresponding to the plurality of medical care providers,
the first common layer to which the state is input thus outputs a feature amount, and
each of the plurality of first individual layers to which the feature amount is input thus outputs a treatment action of the corresponding medical care provider.

12. The medical learning system of claim 11, wherein
the processing circuitry is further configured to:
acquire a third inference model that infers treatment progress of the target patient; and
acquire data inferred by the third inference model as the treatment progress data, wherein
the target patient includes a plurality of patients,
the third inference model includes a second common layer that is common between the plurality of patients, and a plurality of second individual layers respectively corresponding to the plurality of patients,
the second common layer to which the state and a diagnosis and treatment action are input thus outputs a feature amount, and
each of the second individual layers to which the feature amount is input thus outputs a treatment progress of the patient.

13. The medical learning system of claim 12, wherein
the processing circuitry further searches, among the plurality of first individual layers for an optimal first individual layer, for a specific second individual layer of the plurality of second individual layers, or the plurality of second individual layers for a second individual layer optimal for a specific first individual layer of the plurality of first individual layers.

14. The medical learning system of claim 1, wherein
the processing circuitry updates the second inference model based on the treatment progress data at a time point following a time point to which the treatment progress data used in a generation of the second inference model belong.

15. The medical learning system of claim 1, wherein
the processing circuitry manages the second inference model in a block chain.

16. The medical learning system of claim 15, wherein
at a time of inference using the second inference model, the processing circuitry adds the second inference model used in the inference and the treatment progress data to a block, with the second inference model and the treatment progress data being associated with each other.

17. The medical learning system of claim 15, wherein
the processing circuitry is configured to:
update the second inference model based on the treatment progress data relating to a time point following a time point to which the treatment progress data used in a generation of the second inference model belong.
add, at a time of updating the second inference model, the second inference model and the treatment progress data used in the updating, associating the model and the data with each other.

18. The medical learning system of claim 1, wherein
at least one of the target medical care provider or the target patient is a specific individual.

19. A medical learning method comprising:
acquiring a first inference model that infers a treatment action of a target medical care provider based on a state of a patient;
acquiring treatment progress data relating to a target patient; and
generating a second inference model by updating the first inference model based on the treatment progress data relating to the target patient.

20. A non-transitory computer readable storage medium storing a program causing a computer to implement:
acquiring a first inference model that infers a treatment action of a target medical care provider based on a state of a patient;
acquiring treatment progress data relating to a target patient; and
generating a second inference model by updating the first inference model based on the treatment progress data relating to the target patient.
